(19) United States
(12) Patent Application Publication
(10) Pub. No.: US 2006/0247278 A1
(43) Pub. Date: Nov. 2, 2006

(54) Physiologically acceptable salts of 3-[(2-{[4-(hexyloxycarbonylamino-imino-methyl)-phenylamino]-methyl}-1-methyl-1H-benzimidazol-5-carbonyl)-pyridin-2-yl-amino]-propionic acid ethyl ester

(75) Inventors: Peter Sieger, Mittelbiberach (DE); Norbert Hauel, Schemmerhofen (DE); Rolf Schmid, Baltringen (DE)

Correspondence Address: MICHAEL P. MORRIS;BOEHRINGER INGELHEIM CORPORATION, 900 RIDGEBURY ROAD, P. O. BOX 368, RIDGEFIELD, CT, 06877-0368, US

(73) Assignee: Boehringer Ingelheim International GmbH, Ingelheim (DE)

(21) Appl. No.: 11/380,351

(22) Filed: Apr. 26, 2006

(30) Foreign Application Priority Data
Apr. 27, 2005 (DE) 102005020002

(51) Int. Cl. A61K 31/4439; C07D 403/02

(52) U.S. Cl. 514/338; 546/273.4

(57) ABSTRACT

The invention relates to new, physiologically acceptable salts of the active substance ethyl 3-[(2-{[4-(hexyloxycarbonylamino-imino-methyl)-phenylamino]-methyl}-1-methyl-1H-benzimidazole-5-carbonyl)-pyridin-2-yl-amino]-propionate.

### RELATED APPLICATIONS

This application claims priority to German Patent Application No. DE 102005020002.8, filed Apr. 27, 2005, the content of which is incorporated herein by reference in its entirety.

### FIELD OF THE INVENTION

The invention relates to new, physiologically acceptable salts of the active substance ethyl 3-[(2-{[4-(hexyloxycarbonylamino-imino-methyl)-phenylamino]-methyl}-1-methyl-1H-benzimidazole-5-carbonyl)-pyridin-2-yl-amino]-propionate, the enantiomers, the mixtures and the hydrates thereof.

### BACKGROUND OF THE INVENTION

The invention relates to new, physiologically acceptable salts of the active substance ethyl 3-[(2-{[4-(hexyloxycarbonylamino-imino-methyl)-phenylamino]-methyl}-1-methyl-1H-benzimidazole-5-carbonyl)-pyridin-2-yl-amino]-propionate, the enantiomers, the mixtures and the hydrates thereof. This active substance with the chemical formula 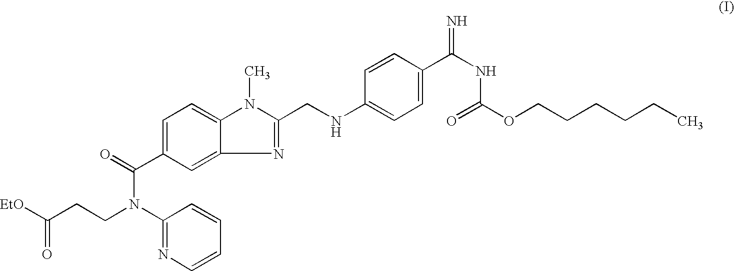
is already known from WO 98/37075, wherein compounds with a thrombin-inhibiting and thrombin time-prolonging activity are disclosed, under the name 1-methyl-2-[N-[4-(N-n-hexyloxycarbonylamidino)phenyl]-amino-methyl]-benzimidazol-5-yl-carboxylic acid-N-(2-pyridyl)-N-(2-ethoxycarbonylethyl)-amide. The compound of formula I is a double prodrug of the compound 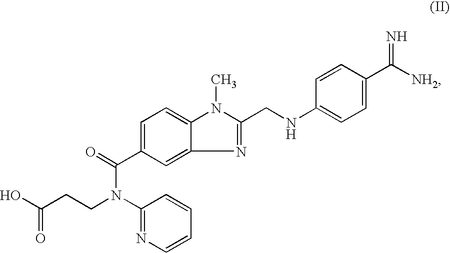
i.e. the compound of formula I is first converted into the actual effective compound, namely the compound of formula II, in the body. The main type of indication for the compound of chemical formula I is the post-operative prophylaxis of deep vein thrombosis and the prevention of strokes.

### DESCRIPTION OF THE INVENTION

The aim of the invention is to prepare new salts of the compound of formula I with advantageous properties for pharmaceutical use.

In addition to being effective for the desired indication, an active substance must also conform to additional requirements in order to be allowed to be used as a pharmaceutical composition. These parameters are to a large extent connected with the physicochemical nature of the active substance.

Without being restrictive, examples of these parameters are the stability of effect of the starting material under various environmental conditions, stability during production of the pharmaceutical formulation and stability in the final medicament compositions. The pharmaceutically active substance used for preparing the pharmaceutical compositions should therefore have a high stability which must be guaranteed even under various environmental conditions. This is absolutely essential to prevent the use of pharmaceutical compositions which contain, in addition to the actual active substance, breakdown products thereof, for example. In such cases the content of active substance in pharmaceutical formulations might be less than that specified.

The absorption of moisture reduces the content of pharmaceutically active substance on account of the weight gain caused by the uptake of water. Pharmaceutical compositions with a tendency to absorb moisture have to be protected from damp during storage, e.g. by the addition of suitable drying agents or by storing the medicament in a damp-proof environment. In addition, the uptake of moisture can reduce the content of pharmaceutically active substance during manufacture if the medicament is exposed to the environment without being protected from damp in any way. Preferably a pharmaceutically active substance should therefore have only limited hygroscopicity.

As the crystal modification of an active substance is important to the reproducible active substance content of a preparation, there is a need to clarify as far as possible any existing polymorphism of an active substance present in crystalline form. If there are different polymorphic modifications of an active substance care must be taken to ensure that the crystalline modification of the substance does not change in the pharmaceutical preparation later produced from it. Otherwise, this could have a harmful effect on the reproducible potency of the drug. Against this background, active substances characterised by only slight polymorphism are preferred.

Another criterion which may be of exceptional importance under certain circumstances depending on the choice of formulation or the choice of manufacturing process is the solubility of the active substance. If for example pharmaceutical solutions are prepared (e.g. for infusions) it is essential that the active substance should be sufficiently soluble in physiologically acceptable solvents. It is also very important for drugs which are to be taken orally that the active substance should be sufficiently soluble.

The problem of the present invention is to provide a pharmaceutically active substance which not only is characterised by high pharmacological potency but also satisfies the above-mentioned physicochemical requirements as far as possible.

Surprisingly it has now been found that the salts of the compound of formula I (dabigatran etexilate) with hydrochloric acid, maleic acid, tartaric acid, salicylic acid, citric acid and malonic acid, the enantiomers, mixtures and hydrates thereof, meet this requirement. Particularly suitable for the purposes of this invention are tartaric acid, salicylic acid and citric acid as well as the enantiomers, mixtures and hydrates thereof.

The following terms are used synonymously:
salt with hydrochloric acid—hydrochloride
salt with maleic acid—maleate
salt with tartaric acid—tartrate
salt with salicylic acid—salicylate
salt with citric acid—citrate
salt with malonic acid—malonate.

The invention therefore relates to the salts of ethyl 3-[(2-{[4-(hexyloxycarbonylamino-imino-methyl)-phenylamino]-methyl}-1-methyl-1H-benzimidazole-5-carbonyl)-pyridin-2-yl-amino]-propionate with hydrochloric acid, maleic acid, tartaric acid, salicylic acid, citric acid and malonic acid as well as the enantiomers, mixtures and hydrates thereof. The invention further relates to pharmaceutical compositions containing at least of one of the above-mentioned salts or hydrates and methods of preparing these pharmaceutical compositions which are suitable for the prevention of venous thromboses and stroke.

The salts according to the invention and also ethyl 3-[(2-{[4-(hexyloxycarbonylamino-imino-methyl)-phenylamino]-methyl}-1-methyl-1H-benzimidazole-5-carbonyl)-pyridin-2-yl-amino]-propionate in the form of the free base and as a salt with methanesulphonic acid are also suitable for the treatment and prevention of deep vein thromboses in patients with heparin-induced thrombocytopenia and for the prevention of thrombosis in patients with intraarterial or intravenous lines or catheters as well as AV shunts.

The melting points were determined by DSC, using an apparatus manufactured by Mettler-Toledo (type: DSC 82 1). The melting temperature used was the onset temperature of the corresponding melting peak in the DSC diagram. The accuracy of the melting points given is about ±3° C.

The starting compound ethyl 3-[(2-{[4-(amino-hexyloxycarbonylimino-methyl)-phenyl-amino]-methyl}-1-methyl-]H-benzimidazole-5-carbonyl)-pyridin-2-yl-amino]-propionate may for example be prepared as described in International Application WO 98/37075, Example 113.

### EXAMPLE 1

### Hydrochloride of ethyl 3-[(2-{[4-(amino-hexyloxycarbonylimino-methyl)-phenylamino]-methyl}--methyl-1H-benzimidazole-5-carbonyl)-pyridin-2-yl-amino]-propionate

125 mg (1.59 mmol) of acetyl chloride were added to 5 ml ethanol with stirring. The solution thus obtained was then added dropwise at ambient temperature to a solution of 1.0 g (1.59 mmol) of ethyl 3-[(2-{[4-(amino-hexyloxycarbonylimino-methyl)-phenylamino]-methyl}-1-methyl-]H-benzimidazole-5-carbonyl)-pyridin-2-yl-amino]-propionate and stirred for a further two hours. The mixture was then evaporated down completely, the residue was first of all triturated after the addition of approx. 5 ml ethyl acetate and suction filtered, then stirred overnight in approx. 10 ml acetone, suction filtered, washed with a little acetone and diethyl ether and then dried at 60° C. in vacuo.

Yield: 86% of theory

Melting point: 135° C.

### EXAMPLE 2

### Citric acid salt of ethyl 3-[(2-{[4-(amino-hexyloxycarbonylimino-methyl)-phenylamino]-methyl}-1-methyl-1H-benzimidazole-5-carbonyl)-pyridin-2-yl-amino]-propionate

210 mg (1.0 mmol) of citric acid hydrate, dissolved in 10 ml ethyl acetate, were added dropwise at ambient temperature with stirring to a solution of 628 mg (1.0 mmol) of ethyl 3-[(2-{[4-(amino-hexyloxycarbonylimino-methyl)-phenylamino]-methyl}-1-methyl-1H-benzimidazole-5-carbonyl)-pyridin-2-yl-amino]-propionate in 45 ml ethyl acetate. A yellow precipitate formed. The mixture was stirred overnight, the product was then suction filtered, washed with a little ethyl acetate and diethyl ether and dried at approx. 50° C. in vacuo.

Yield: 83% of theory

Melting point: approx. 170 ° C. (with decomposition)

### EXAMPLE 3

### Tartaric acid salt of ethyl 3-[(2-{[4-(amino-hexyloxycarbonylimino-methyl)-phenylamino]-methyl}-1-methyl-]H-benzimidazole-5-carbonyl)-pyridin-2-yl-amino]-propionate

150 mg (1.0 mmol) of L(+)-tartaric acid, dissolved in 5 ml absolute ethanol, were added dropwise at ambient temperature with stirring to a solution of 628 mg (1.0 mmol) of ethyl 3-[(2-{[4-(amino-hexyloxycarbonylimino-methyl)-phenylamino]-methyl}-1-methyl-1H-benzimidazole-5-carbonyl)-pyridin-2-yl-amino]-propionate in 50 ml ethyl acetate. A fine precipitate was formed. The suspension was stirred for a further two hours, then the product was suction filtered, washed with a little cold ethyl acetate and diethyl ether and dried in vacuo at approx. 50° C.

Yield: 72% of theory

Melting point: approx. 160° C. (with decomposition)

### EXAMPLE 4

### Malonic acid salt of ethyl 3-[(2-{[4-(amino-hexyloxycarbonylimino-methyl)-phenylamino]-methyl}-1-methyl-]H-benzimidazole-5-carbonyl)-pyridin-2-yl-amino]-propionate

104 mg (1.0 mmol) of malonic acid, dissolved in 10 ml ethyl acetate, were added dropwise at ambient temperature, with stirring, to a solution of 628 mg (1.0 mmol) of ethyl 3-[(2-{[4-(amino-hexyloxycarbonylimino-methyl)-phenylamino]-methyl}-1-methyl-1H-benzimidazole-5-carbonyl)-pyridin-2-yl-amino]-propionate in 50 ml ethyl acetate. After approx. one hour a fine precipitate formed. The suspension was stirred for a further three hours, the product was then suction filtered, washed with a little cold ethyl acetate and diethyl ether and dried in vacuo at approx. 50° C.

Yield: 79% of theory

Melting point: 100° C.

### EXAMPLE 5

### Maleic acid salt of ethyl 3-[(2-{[4-(amino-hexyloxycarbonylimino-methyl)-phenylamino]-methyl}-1-methyl-1H-benzimidazole-5-carbonyl)-pyridin-2-yl-amino]-propionate

116 mg (1.0 mmol) of maleic acid, dissolved in 10 ml ethyl acetate, were added dropwise, with stirring, at ambient temperature, to a solution of 628 mg (1.0 mmol) of ethyl 3-[(2-{[4-(amino-hexyloxycarbonylimino-methyl)-phenylamino]-methyl}-1-methyl-1H-benzimidazole-5-carbonyl)-pyridin-2-yl-amino]-propionate in 50 ml ethyl acetate. A precipitate formed. The suspension was stirred for a further three hours, then the product was suction filtered, washed with a little cold ethyl acetate and diethyl ether and dried in vacuo at approx. 50° C.

Yield: 93% of theory

Melting point: 120° C.

### EXAMPLE 6

### Ethyl-3-[(2-{[4-(hexyloxycarbonylamino-imino-methyl)-phenylamino]-methyl}-1-methyl-1H-benzimidazole-5-carbonyl)-pyridin-2-yl-amino]-propionate salicylate

A solution of 1.38 g (10.0 mmol) of salicylic acid in 20 ml acetone was added dropwise with stirring at 35-40° C. to a solution of 6.28 g (10.0 mmol) of ethyl 3-[(2-{[4-(hexyloxycarbonylamino-imino-methyl)-phenylamino]-methyl}-1-methyl-1H-benzimidazole-5-carbonyl)-pyridin-2-yl-amino]-propionate base (prepared as described in WO 98/37075), in 45 ml acetone. After a few minutes the product began to crystallise out and it was diluted with 65 ml acetone. Within 30 minutes the mixture was cooled to ambient temperature, then the precipitate was suction filtered, washed with approx. 40 ml acetone and dried at 40° C. in the circulating air dryer.

Yield: 94% of theory

Melting point: 155° C.

### EXAMPLE 7

### Dry ampoule containing 75 mg active substance per 10 ml

| | | |
|---|---|---|
| active substance | 75.0 | mg |
| mannitol | 50.0 | mg |
| water for injections | ad 10.0 | ml |

Preparation:

Active substance and mannitol are dissolved in water. After packaging the solution is freeze-dried. To produce the solution ready for use for injections, the product is dissolved in water.

### EXAMPLE 8

Dry ampoule containing 35 mg of active substance per 2 ml

Composition:

| | | |
|---|---|---|
| Active substance | 35.0 | mg |
| Mannitol | 100.0 | mg |
| water for injections | ad 2.0 | ml |

Preparation:

Active substance and mannitol are dissolved in water. After packaging, the solution is freeze-dried.

To produce the solution ready for use for injections, the product is dissolved in water.

### EXAMPLE 9

Tablet containing 50 mg of active substance

Composition:

| | |
|---|---|
| (1) Active substance | 50.0 mg |
| (2) Lactose | 98.0 mg |
| (3) Maize starch | 50.0 mg |
| (4) Polyvinylpyrrolidone | 15.0 mg |
| (5) Magnesium stearate | 2.0 mg |
| | 215.0 mg |

Preparation:

(1), (2) and (3) are mixed together and granulated with an aqueous solution of (4). (5) is added to the dried granulated material. From this mixture tablets are pressed, biplanar, faceted on both sides and with a dividing notch on one side.

Diameter of the tablets: 9 mm.

### EXAMPLE 10

Tablet containing 350 mg of active substance

Composition:

| | |
|---|---|
| (1) Active substance | 350.0 mg |
| (2) Lactose | 136.0 mg |
| (3) Maize starch | 80.0 mg |
| (4) Polyvinylpyrrolidone | 30.0 mg |
| (5) Magnesium stearate | 4.0 mg |
| | 600.0 mg |

Preparation:

(1), (2) and (3) are mixed together and granulated with an aqueous solution of (4). (5) is added to the dried granulated material. From this mixture tablets are pressed, biplanar, faceted on both sides and with a dividing notch on one side.

Diameter of the tablets: 12 mm.

### EXAMPLE 11

Capsules containing 50 mg of active substance

Composition:

| | |
|---|---|
| (1) Active substance | 50.0 mg |
| (2) Dried maize starch | 58.0 mg |
| (3) Powdered lactose | 50.0 mg |
| (4) Magnesium stearate | 2.0 mg |
| | 160.0 mg |

Preparation:

(1) is triturated with (3). This trituration is added to the mixture of (2) and (4) with vigorous mixing.

This powder mixture is packed into size 3 hard gelatine capsules in a capsule filling machine.

### EXAMPLE 12

Capsules containing 350 mg of active substance

Composition:

| | |
|---|---|
| (1) Active substance | 350.0 mg |
| (2) Dried maize starch | 46.0 mg |
| (3) Powdered lactose | 30.0 mg |
| (4) Magnesium stearate | 4.0 mg |
| | 430.0 mg |

Preparation:

(1) is triturated with (3). This trituration is added to the mixture of (2) and (4) with vigorous mixing.

This powder mixture is packed into size 0 hard gelatine capsules in a capsule filling machine.

### EXAMPLE 13

Suppositories containing 100 mg of active substance

| | |
|---|---|
| Active substance | 100.0 mg |
| Polyethyleneglycol (M.W. 1500) | 600.0 mg |
| Polyethyleneglycol (M.W. 6000) | 460.0 mg |
| Polyethylenesorbitan monostearate | 840.0 mg |
| | 2,000.0 mg |

### EXAMPLE 14

| | Percentage composition | | | | | |
|---|---|---|---|---|---|---|
| | | | Active | | per | per |
| | Core | Separating | substance | | capsule | capsule |
| | material | layer | layer | Total | [mg] | [mg] |
| Tartaric acid | 61.3 | — | — | 61.3 | 176.7 | 353.4 |
| Gum arabic | 3.1 | 2.8 | | 5.9 | 17.0 | 34.0 |
| Talc | — | 5.6 | 3.2 | 8.8 | 25.4 | 50.7 |
| Hydroxyhydroxypropyl- | — | — | 4.0 | 4.0 | 11.5 | 23.1 |
| cellulose | | | | | | |
| Active substance (based on | — | — | 20.0 | 20.0 | 50.0 | 100.0 |
| the base) | | | | | | |
| Total | | | | 100.0 | 288.3 | 576.5 |

### EXAMPLE 15

| | Percentage composition | | | | | |
|---|---|---|---|---|---|---|
| | | | Active | | per | per |
| | Core | Separating | substance | | capsule | capsule |
| | material | layer | layer | Total | [mg] | [mg] |
| Tartaric acid | 38.5 | — | — | 38.5 | 55.5 | 166.5 |
| Gum arabic | 1.9 | 1.7 | | 3.6 | 5.2 | 15.6 |
| Talc | — | 3.5 | 6.4 | 9.9 | 14.3 | 42.8 |
| Hydroxyhydroxypropyl- | — | — | 8.0 | 8.0 | 11.5 | 34.6 |
| cellulose | | | | | | |
| Active substance (based on | — | — | 40.0 | 40.0 | 50.0 | 150.0 |
| the base) | | | | | | |
| Total | | | | 100.0 | 144.2 | 432.5 |

The preparation and the structure of the pellets according to Examples 14 and 15 is described in detail in WO 03/074056.

We Claim:

1. Ethyl 3-[(2-{[4-(hexyloxycarbonylamino-imino-methyl)-phenylamino]-methyl}-1-methyl-1H-benzimidazole-5-carbonyl)-pyridin-2-yl-amino]-propionate hydrochloride and the hydrates thereof.

2. Ethyl 3-[(2-{[4-(hexyloxycarbonylamino-imino-methyl)-phenylamino]-methyl}-1-methyl-1H-benzimidazole-5-carbonyl)-pyridin-2-yl-amino]-propionate maleate and the hydrates thereof.

3. Ethyl 3-[(2-{[4-(hexyloxycarbonylamino-imino-methyl)-phenylamino]-methyl}-1-methyl-1H-benzimidazole-5-carbonyl)-pyridin-2-yl-amino]-propionate tartrate and the enantiomers, the mixtures and the hydrates thereof.

4. Ethyl 3-[(2-{[4-(hexyloxycarbonylamino-imino-methyl)-phenylamino]-methyl}-1-methyl-1H-benzimidazole-5-carbonyl)-pyridin-2-yl-amino]-propionate salicylate and the hydrates thereof.

5. Ethyl 3-[(2-{[4-(hexyloxycarbonylamino-imino-methyl)-phenylamino]-methyl}-1-methyl-1H-benzimidazole-5-carbonyl)-pyridin-2-yl-amino]-propionate citrate and the hydrates thereof.

6. Ethyl 3-[(2-{[4-(hexyloxycarbonylamino-imino-methyl)-phenylamino]-methyl}-1-methyl-1H-benzimidazole-5-carbonyl)-pyridin-2-yl-amino]-propionate malonate and the hydrates thereof.

7. A method for prolonging thrombin activity comprising administering to a patient the compound according to any one of claims 1-6.

8. A method for preventing venous thrombosis and stroke comprising administering to a patient in need thereof a compound according to any one of claims 1-6.

9. A pharmaceutical composition comprising a salt according to any one of claims 1-6, optionally together with one or more inert carriers and/or diluents.
